(19) United States
(12) Patent Application Publication
(10) Pub. No.: US 2025/0241689 A1
(43) Pub. Date: Jul. 31, 2025

(54) SPINE STABILIZATION

(71) Applicant: SI-Bone Inc., Santa Clara, CA (US)

(72) Inventors: David W. POLLY, Edina, MN (US); Francois FOLLINI, Austin, TX (US); Paul M. SAND, Redwood City, CA (US); Bret SCHNEIDER, San Jose, CA (US); Derek P. LINDSEY, San Jose, CA (US); Scott A. YERBY, Montara, CA (US)

(21) Appl. No.: 19/024,752

(22) Filed: Jan. 16, 2025

Related U.S. Application Data
(60) Provisional application No. 63/621,539, filed on Jan. 16, 2024.

(51) Int. Cl. A61B 17/70; A61B 17/00; A61B 17/56

(52) U.S. Cl. CPC A61B 17/707; A61B 17/705; A61B 2017/00477; A61B 2017/00862; A61B 2017/564

(57) ABSTRACT

Methods and implantable devices for increasing stability between at least two adjacent vertebrae in a spine of a subject. Methods may include securing an elongate stabilizing member about a first bony structure of a first vertebra, and securing the elongate stabilizing member about a first bony structure of a second vertebra, the second vertebra inferior to the first vertebra. Implantable devices may include an elongate stabilizing member

### CROSS REFERENCE TO RELATED APPLICATIONS

This application claims the benefit of priority of U.S. Provisional Application No. 63/621,539, filed Jan. 16, 2024, the entire disclosure of which is incorporated by reference herein in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

There is a need for methods, devices and systems (optionally stand-alone fasteners) that add stability to two or more motion segments in the spine by limiting or prevent flexion (optionally upper to lower thoracic spine) without the need to fasten to a pedicle screw or a rod.

### SUMMARY OF THE DISCLOSURE

One aspect of the disclosure is a method of increasing stability between at least two adjacent vertebrae in a spine of a subject.

In this aspect, the method includes securing an elongate stabilizing member about a first bony structure of a first vertebra, and securing the elongate stabilizing member about a first bony structure of a second vertebra, the second vertebra inferior to the first vertebra.

In this aspect, the method optionally includes positioning the securing member to extend across a midline of the spine.

In this aspect, the method optionally includes securing a first portion of the stabilizing member relative to a second adjacent portion of the stabilizing member with one or more fastening elements associated with one or more of the first portion or the second portion to prevent the first portion from being dissociated from or moved relative to the second portion.

In this aspect, the elongate stabilizing member is optionally not secured directly to a pedicle screw or a stabilizing rod.

In this aspect, securing the elongate stabilizing member about a first bony structure of a first vertebra optionally comprises at least one of securing the elongate stabilizing member about a spinous process of the first vertebra or securing the elongate stabilizing member within a spinous process of the first vertebra.

In this aspect, securing the elongate stabilizing member about a first bony structure of a second vertebra optionally comprises securing the elongate stabilizing member about or within a spinous process of the second vertebra.

In this aspect, securing the elongate stabilizing member about a first bony structure of a first vertebra optionally comprises securing the elongate stabilizing member about a transverse process of the first vertebra.

In this aspect, securing the elongate stabilizing member about a first bony structure of a second vertebra optionally comprises securing the elongate stabilizing member about a transverse process of the second vertebra.

In this aspect, securing the elongate stabilizing member about a first bony structure of a first vertebra optionally comprises securing the elongate stabilizing member about a lamina of the first vertebra.

In this aspect, securing the elongate stabilizing member about a first bony structure of a second vertebra optionally comprises securing the elongate stabilizing member about a lamina of the second vertebra.

In this aspect, the securing member optionally extends across a midline of the spine and/or optionally within a vertebral foramen of the spine.

In this aspect, the method is optionally performed in a subject in which a laminectomy has been performed.

In this aspect, the first and second vertebrae are optionally thoracic vertebrae.

In this aspect, the subject optionally has an implanted construct rod or is in the process of having a construct rod implanted.

In this aspect, the method is optionally part of one or more of ligament reconstruction, proximal junctional kyphosis (PJK) reduction, or a sublaminar band implantation.

In this aspect, the one or more fastening elements optionally comprise one or more of: a zip-tie, a cam lock, a come-along, ratchet, rack & pinion, Velcro, BOA-like, a knot in the elongate member, a set screw compression, a turnbuckle, or finger trap.

One aspect of this disclosure is a method of limiting flexion between at least two vertebrae in a spine of a subject, and may include any feature of the previous aspect in this Summary section.

In this aspect, the elongate stabilizing member optionally comprises a spring.

In this aspect, the method optionally includes adjusting tension in a spring to further limit flexion between two adjacent vertebrae in the spine of the subject.

One aspect of this disclosure is an implantable spine stabilizing device (a device) that includes an elongate stabilizing member.

In this aspect, the elongate stabilizing member is optionally sized, configured and adapted to be secured about a first bony structure of a first vertebra.

In this aspect, the elongate stabilizing member is optionally sized, configured and adapted to be secured about a first bony structure of a second vertebra, the second vertebra inferior to the first vertebra.

In this aspect, the elongate stabilizing member is optionally sized, configured and adapted to be disposed across a midline of the spine.

In this aspect, the elongate stabilizing member is optionally sized, configured and adapted such that when implanted, the elongate stabilizing member limits flexion between the first and second vertebrae without being secured to a pedicle screw or a stabilizing rod.

In this aspect, the device optionally includes one or more fastening elements associated with one or more of a first portion or a second portion of the stabilizing element to prevent the first portion from being dissociated from or moved relative to the second portion.

In this aspect, the optional one or more fastening elements optionally comprise one or more of a zip-tie, a cam lock, a come-along, a ratchet, a rack & pinion, Velcro, a BOA-like fastener, a knot in the elongate member, a set screw compression, a turnbuckle, or a finger trap.

In this aspect, the device optionally further comprises a tension adjuster configured to adjust tension in the elongate stabilizing member.

In this aspect, the elongate stabilizing member optionally includes or is a spring.

### BRIEF DESCRIPTION OF THE DRAWINGS

FIG. 1 illustrates an exemplary position of an exemplary implantable spine stabilizing device anchored about spinous processes.

FIG. 2 illustrates an exemplary position of an exemplary implantable spine stabilizing device anchored within apertures in spinous processes.

FIG. 3 illustrates an exemplary position of an exemplary implantable spine stabilizing device anchored about transverse processes.

FIG. 4 illustrates an exemplary position of an exemplary implantable spine stabilizing device anchored about transverse processes.

FIG. 5 illustrates an exemplary position of an exemplary implantable spine stabilizing device anchored about transverse processes.

FIG. 6 illustrates an exemplary position of an exemplary implantable spine stabilizing device anchored about transverse processes.

FIG. 7 illustrates an exemplary position of an exemplary implantable spine stabilizing device anchored about transverse processes and spinous processes.

FIG. 8 illustrates an exemplary position of an exemplary implantable spine stabilizing device anchored about transverse processes and within a vertebral foramen.

FIG. 9 illustrates an exemplary position of an exemplary implantable spine stabilizing device anchored about transverse processes and within a vertebral foramen.

FIG. 10 illustrates an exemplary position of an exemplary implantable spine stabilizing device anchored about transverse processes in a subject in which a laminectomy has been performed.

FIG. 11 illustrates an exemplary position of an exemplary implantable spine stabilizing device anchored about at a plurality of laminae of adjacent vertebrae.

FIG. 12 illustrates part of a sublaminar band positioning technique.

FIG. 13 illustrates a band coupled to a rod.

FIGS. 14, 15 and 16 illustrate exemplary pathways and positioning of Luque wires.

FIG. 17 illustrates exemplary positions of cerclage wires, for which the elongate stabilizing members herein may be used as an alternative.

FIGS. 18 and 19 illustrate anatomy and are a reference to spinal anatomy described herein.

### DETAILED DESCRIPTION

The disclosure is related to devices, systems, and methods to add stability between two or more motion segments in the spine. The approaches herein comprise limiting and optionally preventing extension (bending over) between two or more motion segments in the spine.

Examples herein include approaches that do not fasten to existing pedicle screws and rods, which is referred to as being independent, or stand-alone.

In some examples herein the approach includes bilateral placement such that the device or system extends across a midline of the spine.

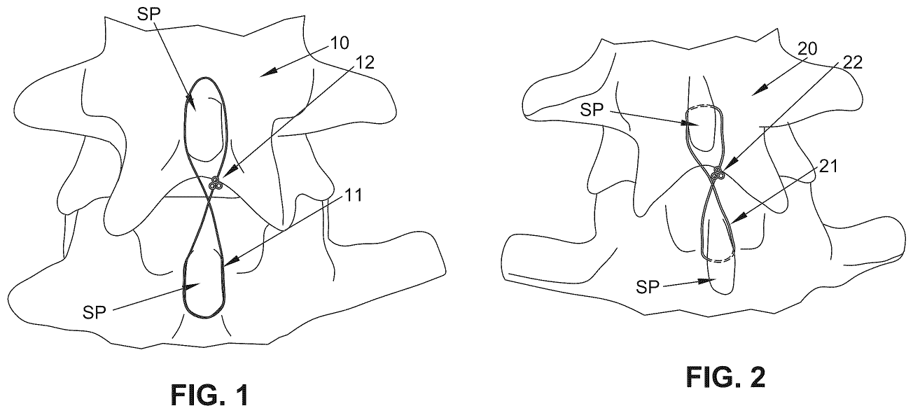

In some examples herein the approach includes a device or system that has a general figure-8 configuration after being implanted, such as is shown in FIGS. 1-3, 4 and 6-11.

One of the benefits of the approaches herein is that they are easy to use and reliable.

One aspect of the disclosure is a method of adding stability between two or more motion segments in the spine that includes limiting or preventing extension. The method may comprise securing an elongate stabilizing member about a first bony structure of a first vertebra and securing the elongate stabilizing member about a first bony structure of a second vertebra, the second vertebra inferior to the first vertebra, where the securing member additionally extends across a midline of the spine. The method may further comprise securing a first portion of the stabilizing member relative to an adjacent second portion of the stabilizing member with one or more fastening elements associated with at least one of the first portion or the second portion to prevent relative motion between the first portion and the second portion.

Securing a first portion of the stabilizing member relative to an adjacent second portion of the stabilizing member with one or more fastening elements may include incremental or progressive tightening or tensioning, wherein incremental or progressive tightening or tensioning increases the stability between the two or more motion segments and further limits flexion. Elongate stabilizing members herein may also be referred to as stabilizing members.

FIGS. 1 and 2 illustrate exemplary implantable spine stabilizing devices 10 and 20, including elongate members 11 and 21, respectively, which in this example are secured about two spinous processes (“SP”) of adjacent vertebrae in a spine, as shown. In this context, each SP may be considered a bony structure anchor location for the elongate stabilizing members 11 and 21. FIG. 2 illustrates elongate member 21 extending through apertures in adjacent SPs, with the dashed lines representing regions of the elongate stabilizing member 21 extending through and within the apertures in the SPs. The apertures may be created through the one or more SPs as part of the procedure using a variety of techniques. Any of the spine stabilizing devices may be referred to herein as implantable devices.

Any of the elongate stabilizing members herein may include at least one portion or section that is wound around portions of the anatomy more than once, which is referred to herein as multiple windings, multi-wound, or similar derivates thereof. For example, elongate stabilizing members herein may include sections wherein the elongate stabilizing member is multi-ply (e.g., two-ply, three-ply) or multi-layered (e.g., two or three layers of material, etc.) along at least a portion of its length when fully positioned. Multiple windings may be needed or desired to facilitate securing a first portion of the stabilizing member to a second portion of the stabilizing member with one or more fastening elements, which may depend on the type of fastening element(s), if the device includes fastening element(s), and/or the anatomical anchor locations of the stabilizing member.

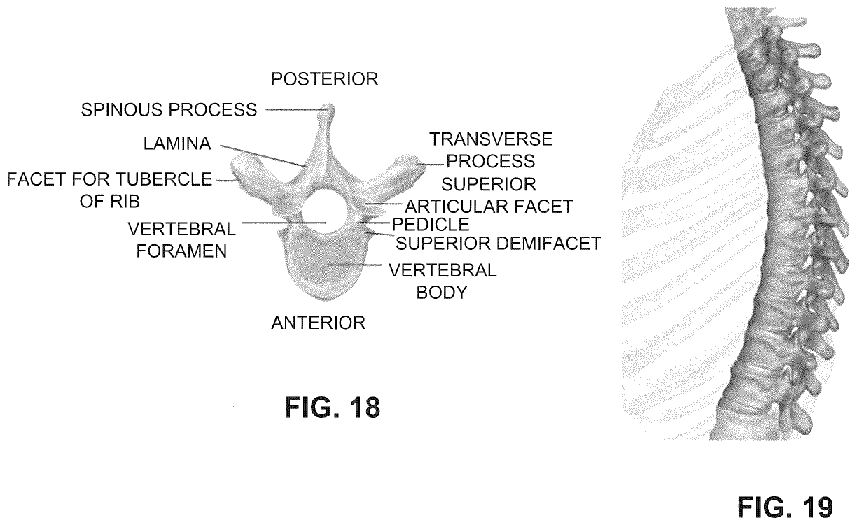

Even if not labeled or described in the text, the vertebrae in the figures herein are understood to include known anatomical structures (e.g., transverse process, lamina, pedicle, etc.), unless specifically indicated otherwise. FIGS. 18 and 19 illustrate exemplary anatomical illustrations, any aspect or portion of which may be incorporated into any of the examples and other figures herein.

Each of the implantable devices 10 and 20 in FIGS. 1 and 2 also include one or more fastening elements 12 and 22, respectively. Fastening elements herein (e.g., 12, 22) are optionally associated with one or more of a first portion or a second portion of the stabilizing member, and can be positioned, sized, and configured to secure or fasten the first portion of the stabilizing member relative to the second portion of the stabilizing member to prevent the first portion from being dissociated or moved relative to the second portion, and thereby add stability between the two motion segments to limit flexion therebetween. Fastening elements herein may include one or more of a button(s), zip tic(s), cam lock(s), come-along(s), ratchet(s), rack & pinion(s), Velcro fastening, BOA closures or BOA-like closures, knot(s), set screw compression, turnbuckle(s), or finger trap(s), or other similar means for securing two portions of the stabilizing member relative to one another. Some devices herein may optionally not include a fastening clement, but may include other features such as a tension adjuster to adjust tension within the stabilizing member (e.g., for a spring or spring-like device).

The implantable devices herein may optionally include a plurality of fastening elements in different regions or sections of the implantable device, or they may have different types of fastening elements in different regions or sections of the implantable device. For example, a first section may include a zip-tie while a different section may include a button or other fastening element. While the one or more fastening elements may be different structures than the stabilizing member, in some examples the fastening element is part of the stabilizing member itself, such as a knot formed in the stabilizing member.

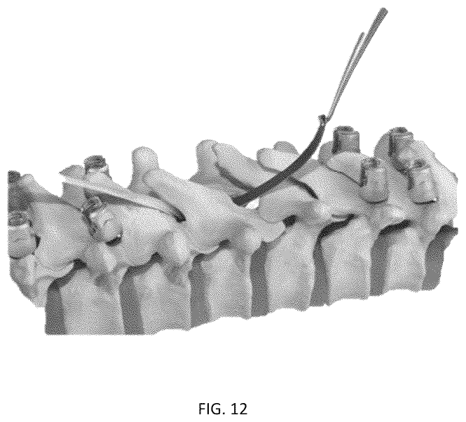

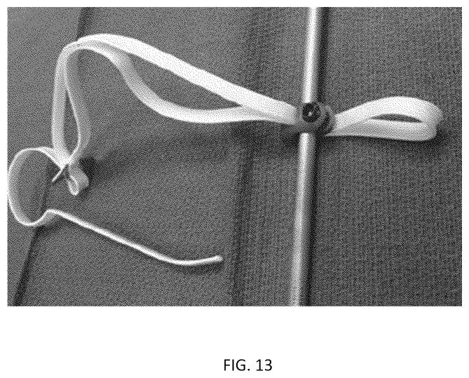

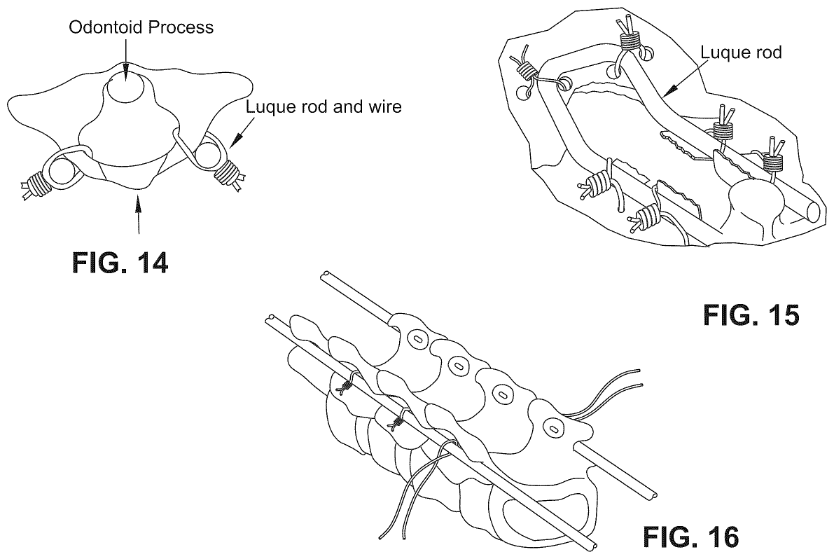

Implantable devices and methods herein may optionally find application with one or more of ligament reconstruction, proximal junctional kyphosis (“PJK”) reduction or sublaminar band (with reference to FIGS. 12 and 13). By way of example only, for PJK, the pathway, or manner in which stabilizing member is positioned, could be similar to a Luque wire, with exemplary reference to FIGS. 14, 15 and 16.

The elongate stabilizing members herein may optionally be a single monolithic component, although in alternative examples the elongate stabilizing member may include a plurality of monolithic components integrated together to form an elongate stabilizing member.

The stabilizing members herein are optionally or preferably made of a material that allows them to be permanently implanted, and optionally or preferably non-resorbable (which may depend on the application). The elongate stabilizing members herein may optionally comprise an elastomeric band. The material is optionally flexible and resistant to elongation, and may optionally include braided or weaved material (e.g., similar to a seat belt). Stabilizing members may comprise a polyester fabric (for example only), such as MERSILENE® tape.

An elongate stabilizing member may optionally comprise one or more springs or devices with spring-like characteristics. The device may further include a tension adjuster that is configured to allow tension in the spring or spring-like device to be adjusted to adjust the stability between the two motion segments as desired.

The stabilizing members herein may be anchored against, or positioned, to interface with one or more different parts of the anatomy, which may be referred to herein as anchor locations. For example, in FIGS. 1 and 2, the device is anchored about/in adjacent spinous processes.

Stabilizing members herein may be anchored at or about one or more bony structures, such as spinous process(es), transverse process(es), lamina, and/or pedicle(s).

In some methods, the implantable devices may be anchored about (including “in”) only spinous processes. In some methods the devices may be anchored about only transverse processes. In some methods the devices may be anchored about only laminae. In some methods the devices may be anchored about only pedicles. In some methods may be anchored about any combination of one or more SPs, one or more transverse processes, one or more laminae, or one or more pedicles.

Devices herein may optionally be anchored about more than one location in a particular vertebra (e.g., SP and transverse process of the same vertebra).

Device herein may optionally be anchored about different locations in adjacent vertebrae. For example only, a stabilizing member may be anchored about transverse processes in a superior vertebra, and about or in a spinous process in an inferior vertebra.

In some methods the stabilizing member may be wound multiple times with multiple windings to facilitate being anchored about one or more different anatomical location in a plurality of vertebrae (e.g., two windings, three winding, one and a half windings, two and a quarter windings, or any other number of windings as needed or desired to perform the procedure for a particular application).

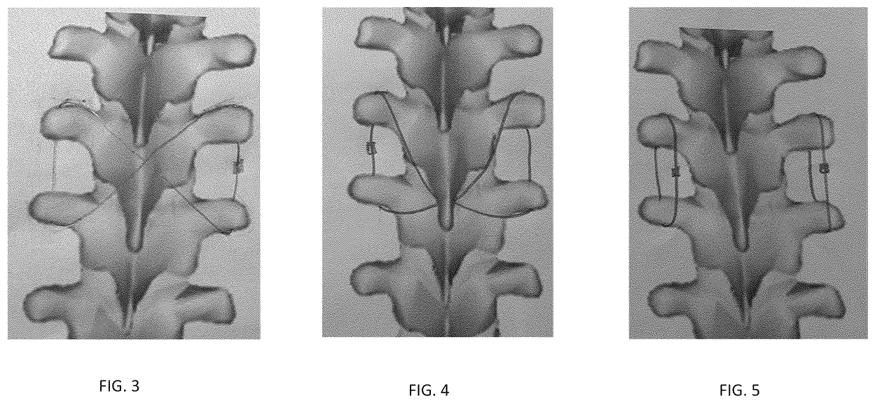

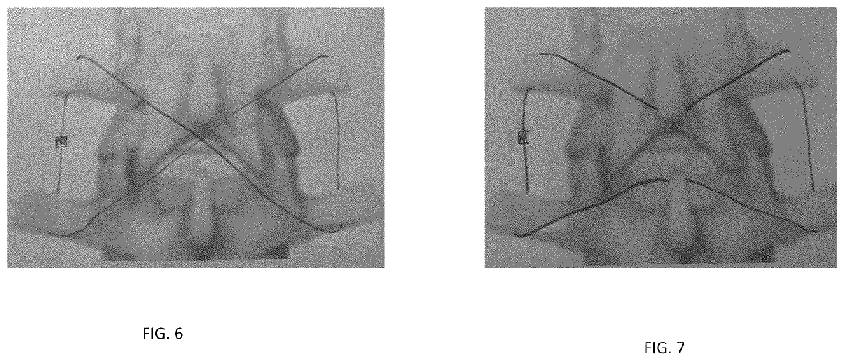

FIGS. 3, 4 and 5 illustrate alternative anchoring locations for stabilizing members that include at least a plurality of transverse processes in adjacent vertebrae, as shown. Illustrative fastening elements are also shown. FIG. 5 is an example of using a plurality of stabilizing members that are not directly coupled to one another. FIGS. 6 and 7 illustrate additional or alternative methods in which stabilizing members may be anchored about a plurality of transverse processes, while the implantable position in FIG. 7 also anchors the stabilizing member about or against spinous processes in the adjacent vertebrae. Illustrative fastening elements and locations are also shown. While only a single winding is illustrated in FIGS. 1-7, alternative approaches may include multiple windings, as described herein. FIGS. 1-7 may include any of the devices herein, any of the stabilizing members herein, as well as any of the optional fastening elements herein.

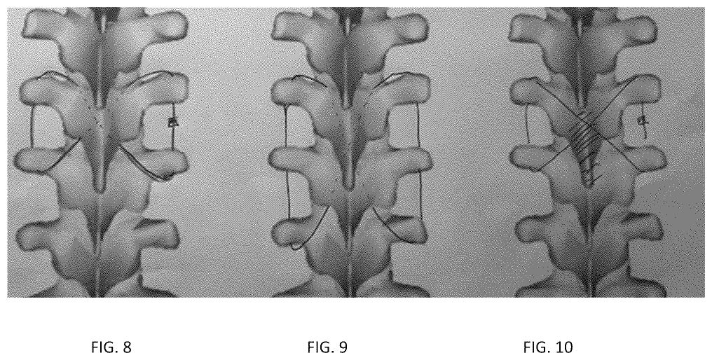

FIG. 8 illustrates an alternative pathway for an exemplary stabilizing member that includes four anchor locations at a plurality of transverse processes (in this example, four transverse processes). FIG. 8 shows the exemplary securing member extending across a midline of the spine and within a vertebral foramen, as indicated by the dashed lines.

FIG. 9 illustrates an alternative pathway for an exemplary stabilizing member that includes four anchor locations at a plurality of transverse processes, wherein the stabilizing members spans more than two vertebrae (three vertebrae in this example). In FIG. 9, the exemplary securing member extends across a midline of the spine and within a vertebral foramen, as indicated by the dashed lines.

FIG. 10 illustrates an alternative pathway for an exemplary stabilizing member that includes four anchor locations at a plurality of transverse processes, wherein the stabilizing member is implanted in a subject in which a laminectomy has been performed (illustrated by the plurality of lines indicating where bone would have been removed).

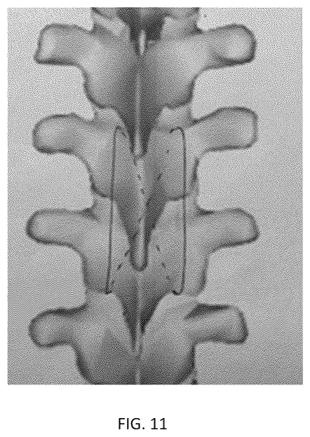

FIG. 11 illustrates an alternative pathway for an exemplary stabilizing member that includes four anchor locations at a plurality of laminae of adjacent vertebrae, as shown. In FIG. 11, the exemplary securing member extends across a midline of the spine and within the vertebral foramen, as indicated by the dashed lines. The device in FIG. 11 may optionally include one or more fastening elements, examples of which are described elsewhere herein.

FIGS. 8-11 may include any of the devices herein, any of the stabilizing members, as well as any one or more of the optional fastening elements herein.

Devices and systems herein may optionally be configured to allow for progressive tightening and holding potential, which may depend on a particular type of fastening element(s).

Stabilizing members herein may have a generally rectangular configuration in two dimensions (length/width), such as a band configuration, or a length of tape. In these examples the stabilizing members may have generally uniform physical properties and characteristics throughout the stabilizing member.

Alternatively, stabilizing member may have one or more dimensions that vary along its length. For example only, a width of the stabilizing member may not be constant or uniform along an entire length of the stabilizing member. A width in one or more sections or regions of the stabilizing member that are adapted to engage or interface with bony surfaces (anchor) may vary from widths of other sections or regions of the stabilizing member, such as if a width is relatively less to reduce stress at the bony interface/anchor or better anchor against the bone structure. Alternatively or additionally, the material of the stabilizing member may vary along a length of the stabilizing member to impart different material properties at different regions along its length. For example only, different sections along its length may not comprise the exact same material. Again, for example only, a material at an anchor location may be different than in a section that is not designed to be anchored against tissue. Alternatively or additionally, a thickness (in the “z”, or third dimension), may not be constant along the length of the stabilizing member. For example only, different sections along its length may not comprise the exact same thickness. Again, for example only, a section at an anchor location may have a different thickness (optionally additionally a different material) than in a sections intended not to anchor against bony structure. In this manner, the physical properties and characteristics within the stabilizing member may be varied as desired, which may depend on the specific application of the stabilizing member.

The devices herein optionally allow a user to apply sufficient tension to resist motion beyond a desired range of motion, yet not too much tension to damage the bone or cause other injury.

Any of the stabilizing members herein (e.g., a band) may optionally include one or more discrete radiopaque markers to monitor integrity of the stabilizing member radiographically. Radiopaque markers may be integrated into the stabilizing member at one or more locations along its length. Optionally, one or more fastening elements may be radiopaque.

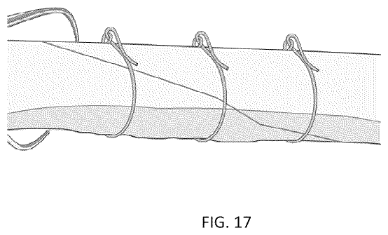

In some applications, the devices herein can find application as a cerclage wire to hold body tissue (e.g., bony segments), together (with reference to cerclage wires shown in FIG. 17).

The devices herein may alternatively or additionally be used to reduce bone fragments. The devices herein may alternatively or additionally be used for ligament reconstruction. The devices herein may alternatively or additionally be used for bony-ligament reconstruction (avulsion). The devices herein may alternatively or additionally be used for patellar tendon reconstruction.

Method herein include accessing the spine, using known medical techniques including making incisions.

Any features described in the context of any examples herein may be combined with other examples unless indicated to the contrary herein.

While the devices and methods herein may find use in the thoracic spine, they may find application in the lumbar spine as well.

FIGS. 21-24 included in U.S. Provisional Application 63/621,539, filed Jan. 16, 2024 (to which this application claims priority and fully incorporates herein) illustrate previous devices, systems and methods that may have features that might be adapted to be (but not necessarily) incorporated into methods, devices and systems herein.

What is claimed is:

1. A method of increasing stability between at least two adjacent vertebrae in a spine of a subject, comprising:
securing an elongate stabilizing member about a first bony structure of a first vertebra;
securing the elongate stabilizing member about a first bony structure of a second vertebra, the second vertebra inferior to the first vertebra,
the securing member further extending across a midline of the spine; and
securing a first portion of the stabilizing member relative to a second adjacent portion of the stabilizing member with one or more fastening elements associated with one or more of the first portion or the second portion to prevent the first portion from being dissociated from or moved relative to the second portion,
wherein the elongate stabilizing member is not secured directly to a pedicle screw or a stabilizing rod.

2. The method of claim 1, wherein securing the elongate stabilizing member about a first bony structure of a first vertebra comprises at least one of securing the elongate stabilizing member about a spinous process of the first vertebra or securing the elongate stabilizing member within a spinous process of the first vertebra.

3. The method of claim 2, wherein securing the elongate stabilizing member about a first bony structure of a second vertebra comprises at least one of securing the elongate stabilizing member about a spinous process of the second vertebra or securing the elongate stabilizing member within a spinous process of the second vertebra.

4. The method of claim 1, wherein securing the elongate stabilizing member about a first bony structure of a second vertebra comprises securing the elongate stabilizing member about or within a spinous process of the second vertebra.

5. The method of claim 1, wherein securing the elongate stabilizing member about a first bony structure of a first vertebra comprises securing the elongate stabilizing member about a transverse process of the first vertebra.

6. The method of claim 5, wherein securing the elongate stabilizing member about a first bony structure of a second vertebra comprises securing the elongate stabilizing member about a transverse process of the second vertebra.

7. The method of claim 1, wherein securing the elongate stabilizing member about a first bony structure of a first vertebra comprises securing the elongate stabilizing member about a lamina of the first vertebra.

8. The method of claim 1, wherein securing the elongate stabilizing member about a first bony structure of a second vertebra comprises securing the elongate stabilizing member about a lamina of the second vertebra.

9. The method of claim 1, wherein the securing member extends across a midline of the spine and within a vertebral foramen of the spine.

10. The method of claim 1, wherein the securing member does not extend within a vertebral foramen of the spine.

11. The method of claim 1, wherein the method is performed in a subject in which a laminectomy has been performed.

12. The method of claim 1, wherein the first and second vertebrae are thoracic vertebrae.

13. The method of claim 1, wherein the subject has an implanted construct rod or is in the process of having a construct rod implanted.

14. The method of claim 1, wherein the method is part of one or more of ligament reconstruction, proximal junctional kyphosis (PJK) reduction, or a sublaminar band implantation.

15. The method of claim 1, wherein the one or more fastening elements comprise one or more of: a zip-tie, a cam lock, a come-along, ratchet, rack & pinion, Velcro, BOA-like, a knot in the elongate member, a set screw compression, a turnbuckle, or finger trap.

16. A method of limiting flexion between at least two vertebrae in a spine of a subject, comprising:
securing an elongate stabilizing member about a first bony structure of a first vertebra;
securing the elongate stabilizing member about a first bony structure of a second vertebra, the second vertebra inferior to the first vertebra,
the securing member further extending across a midline of the spine; and
securing a first portion of the stabilizing member to a second portion of the stabilizing member with one or more fastening elements associated with one or more of the first portion or the second portion to prevent the first portion from being dissociated from or moved relative to the second portion,
wherein the elongate stabilizing member is not secured directly to a pedicle screw or a stabilizing rod.

17. A method of limiting flexion between at least two adjacent vertebrae in a spine of a subject, comprising:
securing an elongate stabilizing member about a first bony structure of a first vertebra; and
securing the elongate stabilizing member about a first bony structure of a second vertebra, the second vertebra inferior to the first vertebra,
the securing member positioned to be extending across a midline of the spine, and wherein the elongate stabilizing member is not secured directly to a pedicle screw or a stabilizing rod.

18. The method of claim 17, wherein the elongate stabilizing member comprises a spring.

19. The method of claim 18, further comprising adjusting a tension in the spring to further limit flexion between the least two adjacent vertebrae in the spine of the subject.

20. An implantable spine stabilizing device, comprising:
an elongate stabilizing member that is sized, configured and adapted to be secured about a first bony structure of a first vertebra, is sized, configured and adapted to be secured about a first bony structure of a second vertebra, the second vertebra inferior to the first vertebra, is sized, configured and adapted to be disposed across a midline of the spine, and is sized, configured and adapted such that when implanted, the elongate stabilizing member limits flexion between the first and second vertebrae without being secured to a pedicle screw or a stabilizing rod.

21. The implantable spine stabilizing device of claim 20, further comprising one or more fastening elements associated with one or more of a first portion or a second portion of the stabilizing element to prevent the first portion from being dissociated from or moved relative to the second portion.

22. The implantable spine stabilizing device of claim 20, wherein the one or more fastening elements comprise one or more of a zip-tie, a cam lock, a come-along, a ratchet, a rack & pinion, Velcro, a BOA-like fastener, a knot in the elongate member, a set screw compression, a turnbuckle, or a finger trap.

23. The implantable spine stabilizing device of claim 20, further comprising a tension adjuster configured to adjust tension in the elongate stabilizing member.

24. The implantable spine stabilizing device of claim 23, wherein the stabilizing member includes or consists of a spring.
